(19) United States
(12) Patent Application Publication
(10) Pub. No.: US 2008/0195116 A1
(43) Pub. Date: Aug. 14, 2008

(54) Circumcision clamp and surgical kit

(76) Inventor: Karim Mansour, San Diego, CA (US)

Correspondence Address: BIOTECH BEACH LAW GROUP , PC, 625 BROASWAY, Suite 1210, SAN DIEGO, CA, 92101, US

(21) Appl. No.: 11/704,804

(22) Filed: Feb. 9, 2007

(51) Int. Cl. A61B 17/32

(52) U.S. Cl. 606/118

(57) ABSTRACT

A circumcision clamp and surgical kit according to the present invention includes a rod, a base, and an actuating structure. The rod is elongated and includes a bell-shaped member as well as an engagement structure. The base of the device includes two arms hinged at their distal ends, and capable of reversible engagement at their proximal ends. When closed, the arms form at least two rimmed apertures, positioned in close proximity are arrayed recesses capable of engaging the actuating structure. The actuating structure includes two members hinged such that when their proximal ends are brought together, their distal ends are opposed. The device of the present invention offers enhanced safety, simplicity, ease of use, and cost effectiveness. The device according to this invention is also part of a pre-sterilized circumcision kit for use during the circumcision procedure.

### TECHNICAL FIELD

The present invention relates generally to surgical clamping devices, and more specifically to a circumcision surgical clamp useful for performing circumcision operations and related surgical kits.

### BACKGROUND OF THE INVENTION

The foreskin or prepuce is a retractable double-layered fold of skin and mucous membrane that covers the glans penis and protects the urinary meatus when the penis is not erect. Often, the foreskin of the penis is removed through a procedure called circumcision.

Because of the widespread use and practice of circumcision, a variety of instruments and techniques have been employed to perform the operation over the years. These instruments and techniques achieve the goal of removing excess foreskin from the penis with reduced trauma, pain, blood loss, and discomfort. It is known in the art relating to circumcision operations to provide a device to securely hold the prepuce prior to, during, and after surgical removal thereof. Certain of these devices are members of a group of circumcision devices referred to as the “bloodless” type. They clamp and squeeze the prepuce to prevent blood flow to the tissue that is to be removed. Because blood flow to the prepuce is stopped, little to no bleeding occurs at the wound site formed by cutting the prepuce. However, these devices can be difficult to use. Many of these clamps require threading both layers of the foreskin through a fixed aperture or port. This technique is difficult to perform, especially for newly admitted physicians. Often only one of the folds is properly threaded through the port, which results in an uneven circumcision.

One such “bloodless” circumcision clamp is disclosed in U.S. Pat. Ser. No. 3,392,728, which includes a base having a single port at the proximal end and a stud bolt positioned at the distal end, a rod-like member having a cross pin at an upper end and a bell shaped member at the lower end, and an arm member pivotally mounted to the base and adapted for engagement with the cross pin at the proximal end and slidably mounted to the stud bolt at the distal end. The majority of the rod-like member is able to pass through the single port, but the lip of the bell is formed large enough to prevent passage through the single port. As a consequence, when the smaller end of the bell member is inserted into the port and the member is drawn through as far as it will go, the lip of the bell presses tightly around the circumference of the port. The arm member is pivotally positioned on the base such that lowering the distal end raises the proximal end. The arm member is raised and lowered by tightening or loosening a nut along the stud bolt. Operation of the device includes determining which device to utilize by evaluating the size of the single port offered by each base, drawing the prepuce over the lip of the of the bell-shaped member, feeding the rod through the single port while keeping the prepuce along the outer portion of the bell, engaging the cross pin, and tightening the nut until the prepuce is sufficiently pinched against the rimmed single port.

Current devices are deficient in several respects; first, the base member includes only a single port, which does not accommodate a wide variety of patients. Therefore the practitioner is required to purchase multiple devices for the range of potential patients. Second, it is often difficult for the practitioner to perform and painful for the subject to undergo the fitting of the prepuce through the single fixed port in the base member. Third, frequently only one of the two folds of the prepuce is correctly inserted through the port resulting in an uneven circumcision. Fourth, it can be awkward for a practitioner to effectively raise the bell member when clamping the prepuce, resulting in uneven clamping due to misalignment. Therefore there remains a need to develop improved devices for performing circumcisions.

### SUMMARY OF THE INVENTION

The present invention addresses the deficiencies inherent to current devices by providing an easier-to-use, more versatile clamp that will reduce the likelihood of uneven circumcisions. More specifically the present invention provides a base that can be closed around the foreskin for effective and precise clamping of the foreskin between the bell-shaped member and rimmed aperture. This configuration permits both folds of the prepuce to be easily clamped. Further, the lower manufacturing cost (when compared to current devices) of the present invention make it ideal for one-time use, especially as a component of a pre-sterilized kit. Alternatively, the simplicity of the design lends itself to re-use following sterilization.

In one aspect of the present invention a circumcision clamp is provided including a rod having a bell-shaped member at one end and a engagement structure at the opposing end, a base having two arms hinged at one end and forming at least two rimmed apertures when in the closed position, and an actuating structure including two elongated members including a handle at one end and at the opposing end one member adapted for engagement with the engagement structure for raising the rod and the other member adapted for placement in a recess or throughbore of the base for stabilizing the device. The bell-shaped member receives the prepuce and the base is capable of closing around the received prepuce for ease of use. The engagement structure engages the actuating structure such that the rod may be lifted when the handle of the actuating structure is squeezed. The actuating structure may be locked in the closed or squeezed position.

The base of the device is bifurcated, with the two resulting arms hinged at one end and capable of reversible engagement to one another at the opposite end. When engaged the two arms are substantially parallel. The reversible-engagement capability of the arms provides a distinct advantage over current technology, as it allows the operator to manipulate the base around the patient, instead of, as is the current practice, drawing the patient through a single port of the base. The diameters of the rimmed apertures are different from one another, though the diameters of the rimmed apertures are in all cases smaller than the largest portion of the bell-shaped member or outer lip. This multiplicity of rimmed apertures enables the device to accommodate patients of various sizes, eliminating the need to provide several different bases, a requirement of previous devices. Positioned in close proximity of each rimmed aperture is a recess or throughbore capable of receiving the distal end of one of the elongated members that form the actuating structure.

The actuating structure includes two members hinged near their midpoints or mid region. Each elongated member has a distal and proximal end, with the proximal ends forming a handle. The two members are constructed such that when the proximal ends are maximally proximate and substantially parallel, the distal ends are maximally opposed. One of the distal ends is formed for placement in the recess or throughbore of the base, and the other distal end is formed to engage the rod's engagement structure. The actuating structure is actuated by squeezing the handle. In some embodiments the actuating structure includes a locking means to lock the actuating structure in the closed or squeezed position.

In another aspect of the present invention a circumcision kit is provided including a circumcision clamp and a medical instrument such as a scalpel. The kit may also include additional surgical instruments such as a hemostat and the like. The kit may be provided as a one time use device or a reusable kit.

In another aspect of the present invention a method of performing a circumcision is provided including providing the circumcision clamp of the present invention, placing the head of a patient's penis in the bell-shaped member, pulling the foreskin over the outer portion of the bell-shaped member, closing the base around the bell-shaped member covered with the foreskin, inserting one of the distal ends of the actuating structure in the recess of the base and engaging the rod's engagement structure with the opposing distal end, squeezing the handle the actuating structure to raise the bell-shaped member and foreskin against the rimmed aperture, and cutting the foreskin above the base.

### BRIEF DESCRIPTION OF THE DRAWINGS

FIG. 1 is a side plan view of the circumcision surgical clamp 10 of the present invention showing the ability of the rod 12 and more specifically the bell-shaped member 14 to be raised and lowered by raising and lowering the engagement structure 16 using the actuating structure 18. FIG. 1A is a lowered configuration showing the the bell-shaped member 14 substantially below the base 20. In FIG. 1B, the surgical clamp 10 is shown in a raised configuration. The proximal ends 22a,b of the actuating structure 18 are maximally proximate when the distal ends 24a,b are maximally opposed. In the raised configuration the bell-shaped member 14 is no longer substantially beneath the base 20.

FIG. 2 depicts various views of the preferred rod 12 of the present invention. FIG. 2A is a front plan view of the rod 12 depicted in FIG. 1 showing the bell-shaped portion 14 at one end and the engagement structure 16 at the opposing end, FIG. 2B is a side elevational view of the rod depicted in FIG. 1, FIG. 2C is back plan view of the rod depicted in FIG. 1 turned on its side and FIG. 2D is a bottom plan view of the rod depicted in FIG. 1 showing the inner region 15 of the bell-shaped member 14, which is fabricated to receive the head of a penis.

FIG. 3A is a top plan view of the base 20 shown in FIG. 1 in the closed position, FIG. 3B is a top plan view of the base 20 in the open position, FIG. 3C is a bottom plan view of the base 20 shown in FIG. 3A and FIG. 3D is an elevated side view of the base shown in FIG. 3A. Three rimmed apertures 26a, 26b and 26c are provided having different diameters from one another. The diameter of rimmed aperture 1 26a shown smaller than rimmed aperture 2 26b, which is shown smaller than rimmed aperture 3 26c. Equal sized recesses 28a,b,c in an arced configuration are also depicted. The base 20 includes two arms 19a and 19b hinged 30 at one end and able to reversibly engage one another 32 at the opposing end. As can be viewed in FIG. 3C, the rimmed apertures 26 are tapered outward towards the bottom of the base 20.

FIG. 4 depicts the preferred configuration of the actuating structure 18 and parts thereof. FIG. 3A is a top plan view of the distal end 24a of the upper member 26a, FIG. 3B is a top plan view of the distal end 24b of the lower member 34b. FIG. 4C is a side elevational view of the actuating structure 18 depicted in FIG. 1. The actuating structure is shown in an open position in FIG. 4C and a closed or squeezed position in FIG. 4D. The actuating structure 18 includes two elongated members 34a and 34b hinged 36 at about their midpoint The proximal end 22 includes a handle 36 and a locking means 38 for locking the upper 34a and lower 34b members in a closed configuration. The distal end 24a of the upper elongated member 34a is adapted for engagement with an engagement structure 16 by providing a hook-like configuration for hooking a cross pin. The distal end 24b of the lower elongated member 34b is adapted for placement in the recess 28.

### DETAILED DESCRIPTION OF THE INVENTION

In contrast to previous circumcision clamps, the present invention includes a device that more easily and accurately clamps the foreskin prior to performing a circumcision. As will be envisioned by one skilled the art to which the present invention belongs, the physician or user is able to clamp the foreskin more effectively by closing the arms of the base around the foreskin instead of relying on the user's ability to thread the foreskin through a fixed aperture. In addition the present invention's use of a hand-actuated mechanism requires less effort by the operator and induces less torsional stress on the device, thereby increasing the safety and efficiency of the operation. This safety and efficiency is further increased by adapting the actuating structure to securely engage both the rod and the base through the use of the rod's engagement structure and the base's recesses and/or throughbores. This invention may be embodied in many different forms and should not be construed as limited to the embodiments set forth herein. Rather, these embodiments are provided so that this application will be thorough and complete, and will fully convey the true scope of the invention to those skilled in the art.

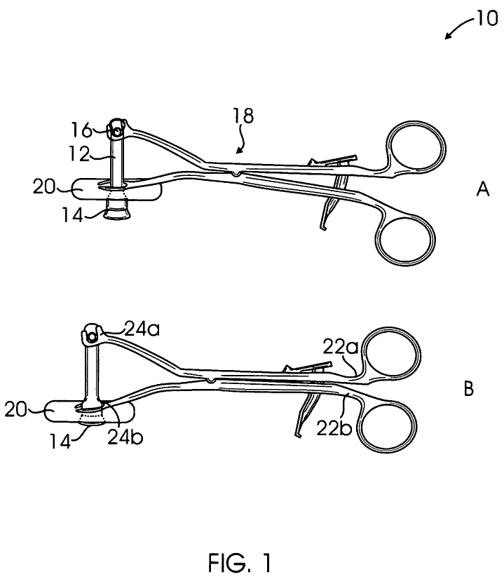

Referring generally to FIGS. 1-4, the circumcision surgical clamp 10 of the present invention includes a rod 12 having at one end an engagement structure 16 and at the opposing end a bell-shaped member 14, a base 20 including two arms 19a and 19b hinged 30 at one end and forming at least two apertures 26 when provided in a closed orientation, and an actuating structure 18 for raising the rod 12 such that the bell-shaped member 14 is raised towards the rim of the rimmed aperture 26. The invention is normally used to perform circumcision operations.

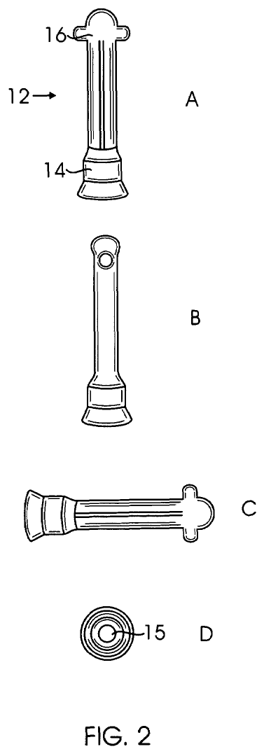

Referring to FIG. 2, at one end of the rod 12, also referred to as the upper end, exists an engagement structure 16. The engagement structure 16 allows the actuating structure 18 to engage the rod 12. The engagement structure 16 may be provided in any configuration that is able to engage an actuating structure 18 such that the actuating structure 18 may exert mechanical force upon the engagement structure 16 and thereby exert such force upon the rod 12, and thus raise the rod 12. Engagement typically involves the mating of two complementary surfaces such as tongue and groove, hook and loop, hook and cross pin, rod and aperture and the like. One skilled the art would be able to develop additional engagement structures 16 using the guidance provided herein, which are also encompassed by the present invention. In the preferred embodiment the engagement structure 16 is in the form of a cross pin. The engagement structure 16 may be fabricated then joined to the rod 12 using conventional casting and joining techniques such as welding, gluing and the like. The engagement structure 16 may also be joined to the rod 12 by non-permanent means, such as by insertion of a pin through a hole or aperture traversing the rod 12. Alternatively the engagement structure 16 may be cast as a single unit (e.g. injection moulded) in combination with the rod 12 using casting methods known to those skilled in the art to which the present invention belongs.

A bell-shaped member 14 is provided at the end opposite the engagement structure 16, also referred to as the lower end of the rod 12. The inner portion 15 of the bell-shaped member 14 accepts the tip of the penis, which allows the foreskin or prepuce to be drawn along the outer portion of the bell-shaped member 14. The bell-shaped member 14 may be fabricated then joined to the rod 12 or may be cast a single piece using methods known to those skilled in the art to which the device 10 of the present invention belongs.

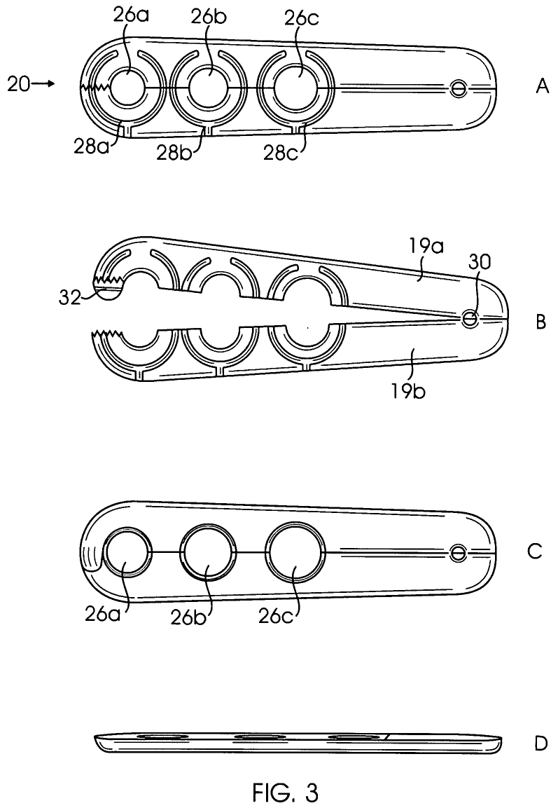

Referring to FIG. 3, the base 20 includes two arms 19a and 19b joined at their distal ends by a hinge 30, which allows the base 20 to reversibly open and close. In some embodiments the base 20 is transparent allowing the practitioner to visually monitor the safety or comfort of the patient or the progress of the procedure. The base 20 provides the primary support for operation of the device 10 and should therefore be constructed from a rigid material. The base arms 19a and 19b may be constructed from metal, metal alloy such as stainless steel or aluminum, plastic, transparent plastic or any other suitable material. The base arms 19 are then hinged 30 at one end using conventional manufacturing techniques such that the base arms 19 are generally planar. The surface of the base 20 may be continuous or discontinuous and may include a variety of surface features such as recesses 28, throughbores, raised planes and the like. In alternative embodiments the hinge 30 is replaced by a pivot pin or a rotatable surface.

When in the closed position, the arms 19 form at least two rimmed apertures 26 having unequal diameters, though the diameters of the rimmed apertures 26 are in all cases greater than the diameter of the elongated portion of the rod 12, and smaller than the largest portion or outer lip of the bell-shaped member 14. Alternative embodiments utilize greater numbers of rimmed apertures 26 such as three, four or more, each having a different diameter, though the diameters of the rimmed apertures are in all cases greater than the diameter of the elongated portion of the rod 12, and smaller than the largest portion of the bell-shaped member 14 of the rod 12. The variation in size of rimmed apertures 26 allows the device to be used with a wide variety of penis sizes, as the variations in diameter size allows the operator to select the most appropriate aperture 26a, 26b or 26c through which to clamp the foreskin, and thus allow a single base 20 to accommodate a multitude of patients. The diameters of the apertures 26 may range from about ¼ inch or about ⅛ inch to about one, two or three inches, with diameters smaller and larger also being encompassed by the present invention. The diameters are preferably different sizes from one another. The rimmed apertures 26 are preferably tapered outwards toward the bottom of the base 20 to at least partially complement a portion of the bell-shaped member 14.

The proximal ends of each of the two base members 19 reversibly engage 32 and may reversibly lock. The reversible engagement 32 or locking may be performed using complementary engaging surfaces or reversibly locking surfaces. As shown in FIG. 3, in one embodiment a locking means is provided having complementary locking angled teeth. In this embodiment the angled teeth allow the proximal ends to be joined by pushing one set of complementary angled teeth over the corresponding second. Release may occur by twisting or lifting one of the arms 19a or 19b generally upwards or downwards relative to the other in order to unlock the teeth, then pulling the proximal ends away from one another. The reversible engagement 32 or locking of the base members 19 ensures a stable surface upon which the bell-shaped member 14 of the rod 12 may exert force by pressing against the base 20 as the actuating structure 18 raises the rod 12 through the aperture 20.

Around or in the vicinity of the rimmed apertures 26 are recesses 28 or throughbores for insertion of the actuating structure 18 (e.g. the distal end 24b of the lower elongated member 34b). Recesses 28 are arranged in the vicinity of the rimmed apertures 26. The recesses 22 may be curved or arced, and may follow a portion of the perimeter of the rimmed aperture 26. In another alternative embodiment, the recesses 28 comprise a single, continuous groove surrounding the rimmed apertures 26. In another alternative embodiment, the recesses 28 are non-linear, such as circular or rectangular. The recesses 28 may be provided in any configuration that is able to receive an actuating structure 18 such that the actuating structure 18 may be securely positioned on the base 20 to raise the rod 12 through the aperture 26. Recesses 28 may be formed by drilling or milling into the base 20 to form a desired shape or depth, or alternatively the base 20 may be cast or injection-molded such that the recesses 28 are thereby created.

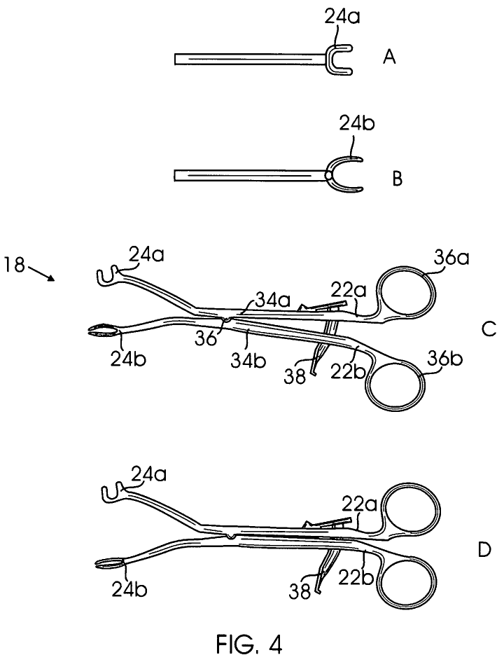

FIG. 4 depicts the preferred embodiment of the actuating structure 18. The actuating structure 18 includes two elongated members 34a and 34b hinged 36 near their midpoints or mid sections. The term “near their midpoints or mid sections” as used herein refers to the portion of the elongated members 34 that is between the distal end 24 and proximal end 22. The members 34 are arranged such that when the proximal ends 22 are maximally proximate and substantially parallel, the distal ends 24 are maximally opposed. The actuating structure 18 may be constructed using methods and materials known in the medical arts such as those used in the construction of hemostats and handheld retractors. As non-limiting examples, the actuating structure 18 may be constructed from metal, metal alloy such as stainless steel or aluminum, plastic and the like.

The proximal ends 22 form a handle 36 allowing the user to squeeze the proximal ends 22 together to raise the rod 12. Each proximal end 22 may be contoured for placement of a finger or thumb such as incorporating a finger loop. The proximal end 22 may further include a locking structure 38 allowing the actuating structure 18 to be locked in the closed or squeezed position such that the distal end 24a of the upper elongated member 34a raises the rod 12. Such locking structures 38 are known in the medical arts, for example those incorporated on hemostats, hand held retractors and the like, which are incorporated by reference and encompassed within the present invention.

One of the members (also referred to as the lower member) 34b is adapted at its distal end 24b for insertion into a recess 28 or throughbore in the base arm(s) 19. The distal end 24b of the lower member 34b stabilizes the actuating structure 18. The adaptation may be provided in a variety of configurations such as having a downward protrusion or a generally arced configuration as provided in FIG. 4 or any other configuration that ensures a secure engagement between the distal end 24b and the recesses 28. The configuration of the lower member's distal end 24b depends on the configuration of the corresponding recess 28 or throughbore. The configuration should be generally complementary. One skilled in the art to which the present invention belongs would recognize that the distal end 24b of the lower member 34b may include a single end for insertion into a single recess 28 or throughbore or a forked end having one or more forks for insertion into two or more recesses 28 or throughbores. The present invention includes a distal end 24b including one or more portions for insertion into one or more recesses 28 or throughbores for stabilization of the present invention. In the preferred embodiment the distal end 24b has an arc-like configuration that is complementary to a recess 28 having an arc-like configuration. In the preferred embodiment the actuating structure 18 is positioned approximately perpendicular to the base 20. Also preferred is a distal end 24b and complementary recess 28 that affect both arms 19 of the base 20 such that the distal end 24b applies force against each of the two arms 19.

The other member (also referred to as the upper member) 34a is adapted at its distal end 24a to engage the engagement structure 16 and thereby manipulate the rod 12 through one of the at least two rimmed apertures 26. The distal end 24a of the upper member 34a is provided in a configuration that is capable of the engagement with the engagement structure 16 such as having a complementary surface. In the preferred embodiments, the upper member 24a is adapted for placement under the engagement structure 16 such as a two pronged hook positionable under a cross pin or under a spherical engagement structure and the like. Therefore in the preferred embodiments the distal end 24a of the upper member 34a is actuated such that it lifts the rod 12 upwards however the present invention also includes embodiments where the upper member 34a is positioned above the rod 12 and pulls the rod 12 upwards. In further embodiments, the actuating structure 18 may include additional features such as an upward protrusion on the lower member to assist in pulling back the foreskin or prepuce for placement around the bell-shaped member.

In another aspect of the present invention a circumcision kit is disclosed including the circumcision clamp 10 of the present invention and one or more surgical tools such as a scalpel, a hemostat, forceps, a dressing, disinfectant, and the like. The kit may be provided as a one time use kit or may be sterilized such as by autoclave one or more times for routine use.

In another aspect of the present invention a method of performing a circumcision is provided including providing the circumcision clamp of the present invention, placing the head of a patient's penis in the bell-shaped member, pulling the foreskin over the outer portion of the bell-shaped member, closing the base around the bell-shaped member covered with the foreskin, inserting one of the distal ends of the actuating structure in the recess of the base and the engaging the rod's engagement structure with the opposing distal end, squeezing the handle the actuating structure to raise the bell-shaped member against the rimmed aperture, and cutting the foreskin above the base.

What is claimed is:

1) A circumcision device comprising:
a) a rod comprising at one end a bell-shaped member and at the opposing end an engagement structure;
b) a base comprising two arms each comprising a distal end and a proximal end, wherein said distal ends are hinged to allow said proximal ends to reversibly open and close, further wherein when in the closed position said two arms form at least two rimmed apertures comprising unequal diameters for receiving said rod, each of said unequal diameters being smaller than the largest portion of said bell-shaped member, said base further comprising a recess capable of receiving an actuating structure; and
c) an actuating structure for raising said rod through one of said at least two rimmed apertures, said actuating structure comprising two elongated members hinged near their midpoints, each of said members having a distal end and a proximal end, said proximal ends together comprising a handle, said distal end of one member adapted for placement in said recess and said distal end of the other member adapted for engagement to said engagement structure, wherein when said actuating structure's proximal ends are substantially parallel, the distal ends are maximally opposed.

2) The circumcision device according to claim 1, wherein said rod, base and actuating structure are constructed from a material selected from the group consisting of stainless steel, aluminum, and plastic.

3) The circumcision device according to claim 1, wherein said engagement structure and said distal end of said actuating structure adapted for engagement comprise complementary engaging structures.

4) The circumcision device according to claim 1, wherein said engagement structure is selected from the group consisting of a cross-pin, a sphere and a throughbore.

5) The circumcision device according to claim 1, wherein said base further comprises a locking means for reversibly locking said arms in said closed position.

6) The circumcision device according to claim 1, wherein said at least two rimmed apertures comprise at least three rimmed apertures, each having a different sized diameter.

7) The circumcision device according to claim 6, wherein said at least three rimmed apertures comprise at least four rimmed apertures, each having a different sized diameter.

8) The circumcision device according to claim 1, wherein said recesses are arced recesses, each positioned generally about the perimeter of one of said at least two apertures.

9) The circumcision device according to claim 8, wherein said distal end adapted for placement in said circular recess is an arced end.

10) The circumcision device according to claim 1, wherein said recesses comprise at least two slotted recesses per aperture.

11) The circumcision device according to claim 1, wherein said recesses are throughbores.

12) The circumcision device according to claim 1, wherein said actuating structure further comprises a locking means capable of locking said actuating structure in a closed position.

13) A prepackaged circumcision kit comprising: the circumcision device according claim 1, and a scalpel.

14) The prepackaged circumcision kit according to claim 12, wherein said circumcision device and said scalpel are sterile.

15) A method of performing a circumcision comprising:
a) providing a circumcision device according to claim 1;
b) inserting a head of a patient's penis within an inner portion of said bell-shaped member;
c) pulling the foreskin over said bell-shaped portion;
d) closing said two arms of said base around the foreskin such that said foreskin is positioned within one of said at least two apertures;
e) placing said distal end of said lower elongated member in said recess;
f) engaging said distal end of said upper elongated member with said engagement structure;
g) squeezing said handle;
h) cutting the foreskin positioned above said base; and
i) releasing said handle and opening said base.
